(19) United States
(12) Patent Application Publication
(10) Pub. No.: US 2006/0259008 A1
(43) Pub. Date: Nov. 16, 2006

(54) Apparatus and methods useful for intravitreal injection of drugs

(75) Inventor: Werhner C. Orilla, Anaheim, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 11/116,695

(22) Filed: Apr. 27, 2005

(51) Int. Cl. A61M 31/00

(52) U.S. Cl. 604/521; 604/239; 604/272

(57) ABSTRACT

Apparatus and methods are provided for delivering therapeutic agents to an eye, for example, into a vitreous of any eye. The apparatus generally includes a conduit coupled to a holder, such as a syringe, containing a therapeutic composition. The conduit includes a distal region having at least one sidewall opening spaced apart from a distal tip and structured to deliver the composition into the vitreous, for example, directly into a peripheral region of the vitreous. The conduit may have a closed distal tip or an open distal tip.

The present invention generally relates to apparatus and methods for delivering therapeutic agents, and more specifically relates to apparatus and methods useful in intraocular delivery of therapeutic agents.

The delivery of drugs to an eye presents many challenges. Topical delivery of drugs often results in limited ocular absorption due to the complex hydrophobic/hydrophilic properties of the cornea and sclera. Diffusion of topically applied drugs to the posterior chamber occurs with some medicinal agents, but often at sub-therapeutic levels.

Local delivery of therapeutics into the vitreous is important in managing several diseases of the eye. Intravitreal injection of therapeutics is an effective means of delivering certain agents to interior portions of the eye. Exceptional care must be taken not injure the lens, retina and other delicate structures of the eye.

Prior art methods of injecting a therapeutic agent such as Triamcinolone Acetonide (Kenalog®) into the vitreous involved using a G27-30 X ¾ inch regular needle attached to a 0.3-0.5 ml syringe. The globe of the eye is stabilized with forceps and the needle is inserted through the conjunctiva and into the vitreous at an angle toward the retina to avoid puncturing the lens. The medicament must be very slowly injected in order to prevent a high pressure jet of the medicament from entering the eye. In addition, some medicaments, such as triamcinolone acetonide which is an opaque fluid, obscure the vision of the patient until the medicament settles to the bottom region of the eye.

It would be advantageous to provide apparatus and methods that provide more accurate intravitreal delivery of therapeutic agents into the eye, for example, to peripheral regions thereof.

### SUMMARY OF THE INVENTION

New apparatus and methods for intravitreal delivery of therapeutic agents to an eye have been discovered.

The present apparatus are structured to more effectively deliver medications into an eye to regions of the eye where the medication is most beneficial. The apparatus also are effective to reduce the occurrence of blurring of the vision after treatment relative to prior art methods and apparatus.

In one broad aspect of the present invention, apparatus are provided for use in delivering a therapeutic agent to an eye. The apparatus generally comprise a conduit, for example, a trocar, cannula, needle or the like, including a proximal portion structured to be coupled to a therapeutic composition holder, for example, a syringe. The conduit further includes a distal portion structured to be placed into a vitreous of an eye. The conduit includes a sidewall and at least one opening in the sidewall. The distal portion of the conduit is structured to deliver the therapeutic composition into the vitreous when the conduit is positioned in the eye with the opening disposed within the vitreous.

In an especially advantageous embodiment, the at least one opening comprises a plurality of openings spaced apart from a distal tip of the conduit.

In some embodiments, the distal tip of the conduit is a closed distal tip. The distal tip may be sharp or beveled or otherwise structured to facilitate placement, for example insertion, of the distal portion of the conduit into the vitreous. In a more specific embodiment, the distal portion includes two openings, hereinafter sometimes “ports”, substantially opposing one another and equidistantly spaced apart from the distal tip.

In another broad aspect of the invention, an apparatus is provided for use in delivering a composition containing a therapeutic agent to a desired location, for example, a peripheral region of an eye. The apparatus generally comprises a conduit including a proximal portion structured to be functionally coupled to a syringe and a distal portion structured to be inserted into a vitreous of an eye. The conduit includes at least one opening in a sidewall thereof and structured to deliver a therapeutic composition into a peripheral region of a vitreous when the conduit is located in the eye with the at least one opening positioned in the vitreous of the eye. As will be shown and described elsewhere herein, by “sidewall opening”, is meant an opening, for example a port, located within a sidewall of the conduit, as opposed to an opening located at a distal tip of the needle. Such a sidewall opening, as used in the present description, is an opening in communication with a longitudinal passageway defined by the conduit. The sidewall opening is spaced apart from the conduit distal tip.

In some embodiments of the invention, the distal portion of the conduit includes a closed distal tip. The distal tip may be sharp or beveled to facilitate insertion thereof into the eye. In a more specific embodiment, the distal portion includes two sidewall openings substantially opposing one another and equidistantly spaced apart from the distal tip.

In yet other embodiments, the distal portion of the conduit includes an open distal tip. In these embodiments, the distal portion of the conduit includes an open tip and at least one lateral port spaced apart from the open distal tip. In a more specific embodiment, the conduit comprises a G27-30 X ¾ inch needle. The conduit includes a distal tip having a beveled opening and two sidewall openings spaced apart from the beveled opening.

In one aspect of the invention, the apparatus further comprises a holder suitable for containing a therapeutic composition and coupleable to the proximal portion of the conduit. For example, the holder may comprise a syringe operably coupled to the proximal portion of the conduit. In another aspect of the invention, the apparatus further comprises a therapeutic composition contained in the holder, the therapeutic composition being useful in treatment of an eye.

Examples of therapeutic compositions useful in the present apparatus and methods include one or more of the following: anti-excitotoxic agents, anti-histamine agents, antibiotic agents, beta blocker agents, one or more steroid agents, anti-neoplastic agents, anti-oxidant agents, anti-inflammatory agents, including non-steroidal anti-inflammatory agents, adrenergic receptor agonists and antagonists, VEGF inhibitor agents, neuroprotective agents, and any ophthalmically therapeutic macromolecule that can be identified and/or obtained using routine chemical screening and synthesis techniques.

In an especially advantageous embodiment, the composition contained in the holder comprises at least one therapeutic agent selected from the group consisting of steroids and steroid precursors. For example, the therapeutic composition comprises at least one steroid selected from the group consisting of cortisone, dexamethasone, prednisolone, prednisolone acetate, triamcinolone, and triamcinolone acetonide.

In some embodiments, the therapeutic composition includes a polymeric component, for example a biodegradable polymeric component or a non-biodegradable polymeric component. The therapeutic composition may include polymeric microparticles and/or polymeric implant elements. In these embodiments and other embodiments of the invention, the openings are suitably sized to allow passage of the polymeric microparticles and or polymeric implant elements from out of the hollow space defined by the conduit 12.

In another aspect of the invention, the conduit is structured to enable a therapeutic composition to be introduced into the eye without any substantial occurrence of jetting of the composition. In addition, in some embodiments, the conduit is structured to reduce turbulence of the composition as the composition is introduced into the eye relative to conventional means of introducing therapeutics into an eye using a conventional needle and syringe.

In yet another aspect of the invention, the conduit is structured to enhance dispersement of the composition as the composition is introduced into the eye.

The present invention further provides methods of delivering therapeutic compositions into an eye, for example, using the apparatus described elsewhere herein, to safely and more effectively deliver therapeutic compositions relative to conventional methods.

Generally, a method in accordance with the invention comprises the steps of providing a therapeutic composition in a holder coupled to a conduit having a sidewall. The conduit includes at least one opening, preferably two or more openings, in the sidewall. The method further includes placing the distal tip of the conduit through the conjunctiva of an eye such that the at least one opening is disposed in the vitreous, and introducing the composition through the at least one opening and into the eye.

In a preferred embodiment, the conduit is structured to be effective to introduce the therapeutic composition in a peripheral region of the vitreous, for example, in an effective location for treating a disorder of the eye and at a distance away from the retina so as to not cause substantial distortion of vision.

In some embodiments, the step of placing the distal tip comprises directing the distal tip of the conduit toward the retina and at an angle effective to avoid contacting the lens of the eye. Preferably, the distal tip is introduced or inserted sufficiently deep such that the at least one sidewall opening is disposed or located within the vitreous. For example, the distal tip of the conduit is inserted in the infero-temporal part of the eye, for example, posterior from the limbus.

The step of introducing the therapeutic composition preferably comprises injecting the therapeutic composition from the holder, for example by means of a plunger disposed in the holder, and out of the conduit through the at least one sidewall opening. In a specific embodiment, the therapeutic composition is introduced directly into a peripheral region of the eye.

In certain advantageous embodiments, the therapeutic composition comprises triamcinolone acetonide, which is an opaque, white medicament that is helpful in treating certain conditions of the eye but has the tendency to blur, block or otherwise distort vision if the medication has been deposited too near the retina.

Any and all features described herein and combinations of such features are included within the scope of the present invention provided that the features of any such combination are not mutually inconsistent.

These and other aspects of the present invention are set forth in the following detailed description and claims, particularly when considered in conjunction with the accompanying drawings in which like parts bear like reference numerals.

### BRIEF DESCRIPTION OF THE DRAWINGS

FIG. 1 is somewhat schematic representation of a PRIOR ART apparatus and method of delivering therapeutics into an eye.

FIG. 2 is a somewhat schematic representation of an apparatus, in accordance with an embodiment of the invention, being used to more effectively deliver therapeutics into an eye, relative to the PRIOR ART apparatus shown in FIG. 1.

FIG. 3 is a somewhat schematic representation, similar to FIG. 2, of an alternate apparatus in accordance with the invention.

### DETAILED DESCRIPTION

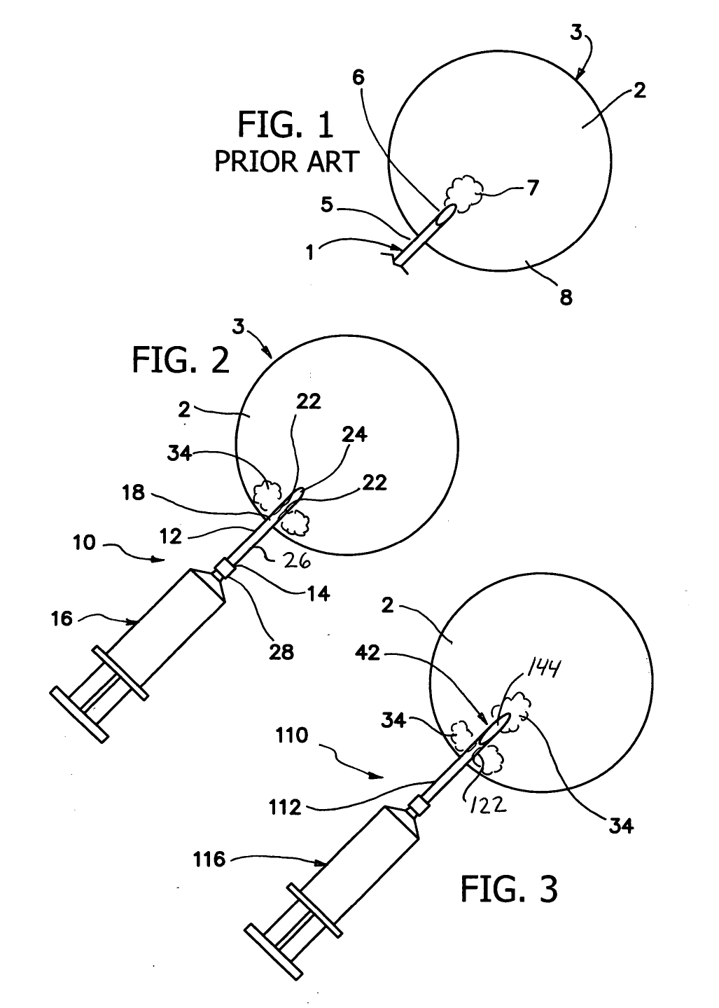

With reference to FIG. 1, a simplified schematic representation of PRIOR ART apparatus 1 being used to deliver a therapeutic composition, for example a medication, for example, triamcinolone acetonide, for example Kenalog®, into the vitreous 2 of an eye 3 is shown. The apparatus 1 is a standard G27-30 X ¾ inch regular needle 5 coupled to a 0.3-0.5 ml syringe (not shown) containing about 0.3 to 0.5 ml of the therapeutic composition.

The general PRIOR ART methodology involves preparing the eye 3 with a disinfectant and/or an anesthetic, stabilizing the globe of the eye 3, for example, using forceps, and inserting the distal tip 6 of the needle 5 into the vitreous at an angle toward the retina. The agent 7 is carefully injected toward the center of the eye 3. Care is taken to inject the agent 7 in a slow and steady manner so as to avoid jetting a high pressure stream of the fluid composition 7 toward the retina which could cause damage thereto. In addition, the amount of agent 7 injected into the eye 3 is kept at a minimum in order to lessen the amount of the agent that might remain in front of the retina for a prolonged period of time. The composition 7 will eventually settle to the bottom 8 of the eye, where it is most effective in providing therapeutic benefit thereto.

Typically, patients who undergo this procedure for introducing a medication containing triamcinolone acetonide are informed that the medication injected into the eye, which is opaque and white in color, will block vision and/or floaters thereof may cause temporary blurring of vision for about a week until the medication settles to the bottom of the eye.

Turning now to FIG. 2, an apparatus in accordance with an embodiment of the invention is shown generally at 10. The apparatus 10 is useful for delivering a therapeutic composition, for example a composition containing a therapeutic agent, for example, but not limited to triamcinolone acetonide, to an eye 3. Generally, the apparatus 10 comprises a conduit 12 including a proximal portion 14 structured to be coupled to a therapeutic composition holder 16, and a distal portion 18 structured to be placed in a vitreous 2 of an eye 3. The distal portion 18 includes at least one opening 22, for example, two openings, spaced apart from a distal tip 24 of the needle 12. The openings 22 are disposed within a sidewall 26 of the conduit 12.

The conduit 12 preferably has size, or outer diameter suitable for the purposes of which the apparatus is intended. In a preferred embodiment of the invention, the conduit 12 has an outer diameter of about 1 mm or less. For example, the conduit 12 may have a gauge as large as G15 to as small as G31. In a preferred embodiment, the conduit has a gauge of between about G30 and about G20. For example, the conduit 112 has a gauge of up to about G22, for example, about G27.

The proximal portion 14 preferably includes a hub 28 or other structure suitable for coupling the conduit 12 to the holder 16. In a specific embodiment, the holder 16 comprises a syringe, for example, a 0.3 to 0.5 ml syringe, useful for containing a therapeutic composition to be introduced into an eye.

In a preferred embodiment of the invention, the distal portion 18 of the conduit 12 is positioned and structured to facilitate delivery of the composition from the conduit 12 away from the center of the eye 3, for example, into a peripheral region of the vitreous where the therapeutic agent will provide the most benefit to the eye and will be least detrimental to vision.

For example, in the embodiment of the invention shown, the openings 22 are located in substantially opposing positions, and are equidistantly spaced apart from the distal tip 24. The substantially opposing openings 22 define, therefore, a single through hole somewhat perpendicular to a longitudinal axis defined by the sidewall 26 of the of the conduit 12.

In a preferred embodiment, the openings 22 are spaced apart from the distal tip 24 a distance in a range of between about 0.5 mm and about 10 mm. In more preferred embodiment, the openings 22 are spaced apart from the distal tip 24 about 1.0 mm, about 0.5 mm, about 0.25 mm, or less.

By comparing FIG. 2 with FIG. 1 (PRIOR ART), it can be appreciated that the positioning of the openings 22 on the sidewall 26 of the conduit distal portion 18 allows for a therapeutic agent 34 to be delivered closer to the periphery of the vitreous 2 relative to a conventional needle in the same position within the eye but not having the ports 22.

In the embodiment shown in FIG. 2, the distal portion 18 includes a closed distal tip 24, for example a closed, angled distal tip for facilitating ease of insertion. It can be appreciated by referring to FIG. 2 that in this specific embodiment, the agent delivered into the eye in being delivered in a direction generally within or near the peripheral region of the eye rather than the center region of the eye. In this specific example, by using the apparatus 10 as shown, none of the therapeutic composition is directly injected into the center region of the eye toward the retina. Some of the advantages of this design over the prior art include faster, more accurate delivery of agent to the site of the eye where it will be most beneficial, less chance of the agent being deposited in front of the retina and obscuring vision, and less chance of the injected agent being “jetted” directly toward the retina.

Turning now to FIG. 3, another apparatus 110 in accordance with the invention is shown. Except as expressly described herein, apparatus 110 is similar to apparatus 10 and features of apparatus 110 which correspond to features of apparatus 10 are designated by the corresponding reference numerals increased by 100.

Apparatus 110 is substantially the same as apparatus 10, with the primary difference being that instead of closed distal tip 24, conduit 112 of apparatus 110 comprises an open distal tip 42. The conduit 112 may comprise a hollow needle, for example a hollow needle having a beveled distal tip 42.

The conduit 112 preferably has size and/or flexibility suitable for the purposes of which the apparatus is intended for use. In a specific embodiment of the invention, the conduit 112 comprises a needle having a gauge of about G22. In a preferred embodiment, the conduit 112 comprises a needle having a gauge of about G27.

As shown, the composition 34 injected into the vitreous 2 is introduced from lateral ports 122 as well as distal port 44. The needle 112 is located in substantially the same position as conduit 12 in the eye 3, with the distal tip 34 pointed toward the center of the eye 3.

Although some of the composition 34 in directed in a direction toward the retina, as shown, the provision of the two lateral ports 122 and the distal opening 44 functions to disperse pressure exerted by the composition during the injection. Thus, it can be appreciated that with this structure, there is less chance, or substantially no chance, of undesirable jetting of composition, which could injure delicate structures of the eye, relative to prior art apparatus.

In accordance with some embodiments, the invention further provides apparatus 10 and 110 having a therapeutic composition holder 16, 116, respectively, containing a therapeutic composition.

Therapeutic compositions which may be provided in the present apparatus 10, 110 in accordance with the invention are compositions which may be obtained from public sources or may be synthesized using routine chemical procedures known to persons of ordinary skill in the art. Such compositions are screened for therapeutic efficacy using conventional assays known to persons of ordinary skill in the art. For example, compositions can be monitored for their effect in reducing intraocular pressure, reducing or preventing neovascularization in the eye, reducing inflammation in the eye, and the like, using conventional assays. Thus, the present systems can compromise a variety of therapeutic compositions.

Specific examples of therapeutic compositions and agents contained in these compositions, which are useful in the present systems include one or more of the following: anti-excitotoxic agents, anti-histamine agents, antibiotic agents, beta blocker agents, one or more steroid agents, anti-neoplastic agents, anti-oxidant agents, anti-inflammatory agents, including non-steroidal anti-inflammatory agents, adrenergic receptor agonists and antagonists, VEGF inhibitor agents, neuroprotective agents, and any ophthalmically therapeutic macromolecule that can be identified and/or obtained using routine chemical screening and synthesis techniques.

In some embodiments, the therapeutic composition includes a polymeric component, for example a biodegradable polymeric component or a non-biodegradable polymeric component. More specifically, the composition may include polymeric microparticles and/or polymeric implant elements.

Although only conduits 12 and 112 having two opposing lateral ports 22 and 122, respectively, are shown, it is to be appreciated that only one lateral port, or more than two lateral ports, may be provided within the scope of the present invention. In addition, the port or ports may be located at various positions along the surfaces of the needle. For example, rather than the substantially opposing positions of ports 22, lateral ports may be provided which are offset from one another. Further, multiple ports may be provided which are equidistantly spaced from the distal tip with the ports being clustered together on only one side of the needle. Ports of various sizes may be provided as well.

Appropriate selection of a number and/or positioning of lateral ports may be dependent upon many factors, including but not limited to the viscosity and/or type of composition to be introduced into the eye, the desired treatment objectives, and/or other parameters that may determine the most beneficial number and/or positioning of lateral ports.

The present invention also provides methods of delivering therapeutic compositions, for example compositions containing therapeutic agents, into an eye. The methods generally comprise the steps of providing a therapeutic composition in a holder, for example a syringe, coupled to a conduit wherein the conduit comprises a needle including at least one sidewall opening spaced apart from a distal tip of the needle. For example, apparatus 10 or 110 are useful in the accomplishing the methods of the present invention.

The methods further include inserting the distal tip of the needle through the conjunctiva of an eye such that the at least one opening is positioned in the vitreous, and introducing the therapeutic agent through the at least one opening and into the eye.

The following, non-limiting Example illustrates certain aspects of the present invention.

### EXAMPLE

A 45 year old woman has been diagnosed with acute inflammation of the vitreous of her left eye. The woman's physician determines that the eye will best respond to treatment with triamcinolone acetionide deposited into the bottom, peripheral region of the eye.

A needle, having the size and shape of a G27-30 X ¾ inch regular wall needle, with the modification of the inclusion of two substantially opposing lateral ports, is provided. The needle may therefore be a needle such as shown in FIG. 2 and described elsewhere herein. The needle is coupled to a 0.5 ml syringe containing 2 ml of triamcinolone acetionide.

The eye is prepared by applying proparacaine 0.5% as a topical anesthetic and ocular Betadine 5% as a disinfectant. The site of injection is located on the infero-temporal part of the globe, at 4 mm posterior from the limbus. The globe of the eye is stabilized with forceps and the needle is inserted halfway through the conjunctiva into the vitreous at an angle toward the retina. The needle is directed toward the retina in order to avoid the lens of the eye. 0.1 ml of triamcinolone acetionide is slowly injected into the eye.

The patient is informed that although it is not a certainty, it is possible that some of the medication may cause floaters which will temporarily blur vision for about a week.

However, because the composition was injected by means of apparatus in accordance with the invention, the patient does not experience such floaters and is able to see clearly immediately after the treatment. In addition, because the therapeutic agent was deposited directly at the peripheral portion of the bottom of the eye, the patient experiences more rapid relief of symptoms relative to patients with similar diagnoses who have been treated with standard methodology using standard, conventional needle and syringe.

While this invention has been described with respect to various specific examples and embodiments, it is to be understood that the invention is not limited thereto and that it can be variously practiced within the scope of the following claims.

What is claimed is:

1. An apparatus for use in delivering a therapeutic agent to an eye, the apparatus comprising:
a conduit including a proximal portion structured to be coupled to a holder containing a therapeutic agent and a distal portion having a sidewall and structured to be placed into a vitreous of an eye, and including at least one opening in the sidewall structured to deliver a therapeutic agent from the conduit into the vitreous when the distal portion is located in the vitreous.

2. The apparatus of claim 1 wherein the distal portion includes a closed distal tip.

3. The apparatus of claim 1 wherein the distal portion includes an open distal tip.

4. The apparatus of claim 1 wherein the distal portion includes a beveled distal tip.

5. The apparatus of claim 1 wherein the at least one opening comprises two openings.

6. The apparatus of claim 5 wherein the two openings are defined in substantially opposing surfaces of the sidewall of the distal portion.

7. The apparatus of claim 1 wherein the conduit comprises a needle.

8. The apparatus of claim 7 wherein the needle has a gauge of less than about G20.

9. The apparatus of claim 7 wherein the needle has a gauge of about G30 to about G20.

10. The apparatus of claim 2 wherein the at least one opening comprises two openings substantially equidistantly spaced apart from the closed distal tip.

11. The apparatus of claim 5 wherein the at least one opening comprises two openings substantially equidistantly spaced apart from the open distal tip.

12. The apparatus of claim 1 further comprising a holder coupled to the proximal portion of the conduit.

13. The apparatus of claim 12 wherein the holder comprises a syringe.

14. The apparatus of claim 12 wherein the holder contains a therapeutic agent.

15. The apparatus of claim 14 wherein the therapeutic agent comprises at least one steroid selected from the group consisting of cortisone, dexamethasone, prednisolone acetate, triamcinolone, and triamcinolone acetonide.

16. A method of delivering a therapeutic agent into an eye, the method comprising:
providing a holder containing a composition comprising a therapeutic agent, and a conduit coupled to the holder, the conduit having a sidewall and distal tip and at least one opening in the sidewall spaced apart from the distal tip;
placing the distal tip in an eye such that the at least one opening is located in the vitreous of the eye;
introducing the composition from the holder through the at least one opening and into the eye.

17. The method of claim 16 wherein the step of placing comprises directing the distal tip of the conduit toward the retina of the eye.

18. The method of claim 16 wherein the step of placing comprises inserting the distal tip in the infero-temporal part of the eye, posterior from the limbus.

19. The method of claim 16 wherein the conduit comprises a needle.

20. The method of claim 16 wherein the therapeutic agent comprised at least one steroid selected from the group consisting of cortisone, dexamethasone, prednisolone acetate, triamcinolone, and triamcinolone acetonide.

21. The method of claim 16 wherein the composition further comprises a polymeric component.

22. The method of claim 16 wherein the composition further comprises a biodegradable polymeric component.

23. The method of claim 16 wherein the composition further comprises a non-biodegradable polymeric component.

24. The method of claim 16 wherein the composition includes polymeric microparticles.

25. The method of claim 16 wherein the composition includes polymeric implant elements.

26. An apparatus for use in delivering a therapeutic agent to an eye, the apparatus comprising:
a syringe structured to contain a therapeutic agent useful for treating an eye; and
a hollow needle including a proximal portion structured to be coupled to the syringe and a distal portion having a sidewall and structured to be inserted into a vitreous of an eye and including at least one opening in the sidewall spaced apart from a distal tip of the needle.

27. The apparatus of claim 26 wherein the distal portion is structured to disperse pressure of therapeutic agent injected into an eye from the syringe through the conduit and out of the at least one opening.

28. The apparatus of claim 26 wherein the at least one opening comprises at least two substantially opposing openings.

29. The apparatus of claim 26 wherein the distal tip comprises a closed distal tip.

30. The apparatus of claim 26 wherein the distal tip comprises an open distal tip.

31. The apparatus of claim 26 further comprising a therapeutic agent contained in the syringe.

32. The apparatus of claim 31 wherein the therapeutic agent comprises at least one steroid selected from the group consisting of cortisone, dexamethasone, prednisolone acetate, triamcinolone, and triamcinolone acetonide.
